(19) United States
(12) Patent Application Publication
(10) Pub. No.: US 2006/0281990 A1
(43) Pub. Date: Dec. 14, 2006

(54) User interfaces and navigation methods for vascular navigation

(76) Inventors: Raju R. Viswanathan, St. Louis, MO (US); Jeffrey M. Garibaldi, St. Louis, MO (US)

Correspondence Address: Bryan K. Wheelock, Suite 400, 7700 Bonhomme, St. Louis, MO, 63105, US

(21) Appl. No.: 11/429,668

(22) Filed: May 5, 2006

Related U.S. Application Data
(60) Provisional application No. 60/678,321, filed on May 6, 2005.

(51) Int. Cl. A61B 5/05

(52) U.S. Cl. 600/424

(57) ABSTRACT

A method of controlling a remote navigation system that remotely orients the distal end of the medical device in order to navigate a medical device through a body lumen includes displaying an endoluminal image of the portion of the body lumen through which the device is being navigated, including an image of the distal end of the medical device; displaying a plurality of directional controls associated with the displayed endoluminal image; and accepting inputs of a selected direction of change of orientation of the distal tip from the directional controls and in response operating the remote navigation system to change the direction of orientation of the distal end of the medical device in the selected direction. The distal end of the device may alternatively or additionally be oriented to point toward a point corresponding to a point that the user identifies on the displayed endoluminal image.

### CROSS-REFERENCE TO RELATED APPLICATIONS

This application claims the benefit of U.S. Provisional Patent Application Ser. No. 60/678,321, filed May 6, 2005, the entire disclosure of which is incorporated herein by reference.

### BACKGROUND OF THE INVENTION

This invention relates to the navigation of medical devices through body lumens and cavities, and in particular to an interface for controlling remote navigation systems for navigating medical devices through body lumens and cavities.

Remote navigation systems have been developed which allow a user to remotely orient the distal end of a medical device and thereby navigate the device through a subject's body lumen or cavity, and particularly the subject's vasculature. In particular, magnetic navigation systems have been developed by Stereotaxis, Inc., that apply a strong magnetic field in a selected direction to orient the distal end of a medical device provided with a magnetically responsive element. These magnetic navigation systems provide fast and accurate remote control over the distal end of the medical device. Other attempts have been made to provide remotely navigable medical devices, including devices employing conventional pull wires and push wires, and other mechanical means for remotely orienting the distal end of a medical device. Thus, while the technology is available to remotely navigate medical devices, the in certain circumstance it can be difficult for the physician or other health care worker to visualize the procedure site, and more specifically to indicate to the remote navigation system the desired direction of orientation of the distal end of the medial device.

### SUMMARY OF THE INVENTION

Embodiments of the present invention provide an interface to facilitate the control of medical devices and in particular the control of remotely controlled medical devices. Generally, the interface of the present invention comprises a display of a view from inside the body lumen or cavity (an “endoluminal view”) in the vicinity of the distal end of the medical device. This view may be an actual image from inside the body lumen or cavity, but in the preferred embodiment, it is a reconstructed view from preoperative or intraoperative imaging. This view preferably includes an image of the distal end portion of the medical device. The image of the distal end portion of the medical device may be an actual image of the distal end portion obtained with or separately from the image of the body lumen or cavity. The image of the distal end portion of the medical device is preferably a generated image of the device based upon a model of the device and the current state of the remote navigation system. This combined view of the body lumen and cavity allows the physician or other user more easily understand the current position and orientation of the medical device, and to determine the desired new direction of orientation.

In a first preferred embodiment, a plurality of control buttons are associated with the displayed image. These control buttons can be physical buttons, they can be “virtual” buttons on which the physician or other user can point with a cursor or other indicator and “click”, or they can be defined locations on a touch screen display which the user can operate by touching either with a finger or a stylus. In the preferred embodiment these buttons are arranged around the periphery of the image, and their positions indicate the direction they control. They may also be shaped to visually reinforce the direction associated with the particular button. If the user desires to re-orient the tip of the device in a particular direction, the user simply operates the corresponding button. The displayed image of the medical device updates as the remote navigation system changes the orientation of the distal end portion of the medical device. The buttons could operate in a discrete mode where each operation or “click” changes the orientation in the selected direction by a predetermined amount, or the buttons could operate in a continuous mode where the direction changes as long as the button is held down.

In a second preferred embodiment, the surface of the displayed endoluminal image is active, and when the physician or user identifies a particular point on the image, the remote navigation system orients the distal tip of the device to point toward the selected point. The active surface can be one in which the user points and clicks a cursor, or alternatively it can be touch screen on which the user indicates the desired direction with a finger or a stylus.

In a third preferred embodiment, both the buttons of the first preferred embodiment and the active image of the second preferred embodiment are provided to provide dual modes of control of the remote navigation system.

Thus, the interface and control methods of the various embodiments of the present invention allows the user to visualize and to control the orientation of a distal end of the medical device as it is being navigated in a body lumen or cavity. The interfaces and controls allows the user to more quickly and easily indicate to a remove navigation system the desired orientation of the medical device to facilitate the navigation of a medical device through the body lumen or cavity.

### BRIEF DESCRIPTION OF THE DRAWINGS

FIG. 1 is a view of a computer screen illustrating one possible implementation of the interface and methods of the present invention;

FIG. 2 is a view of a computer screen illustrating a second possible implementation of the interface and methods of the present invention;

FIG. 3 is a view of a first preferred embodiment of an interface implementing a first control method in accordance with the principles of this invention;

FIG. 4 is a view of a second preferred embodiment of an interface implementing a second control method in accordance with the principles of this invention;

FIG. 5 is a view of a third preferred embodiment of an interface implementing a third control method in accordance with the principles of this invention; and

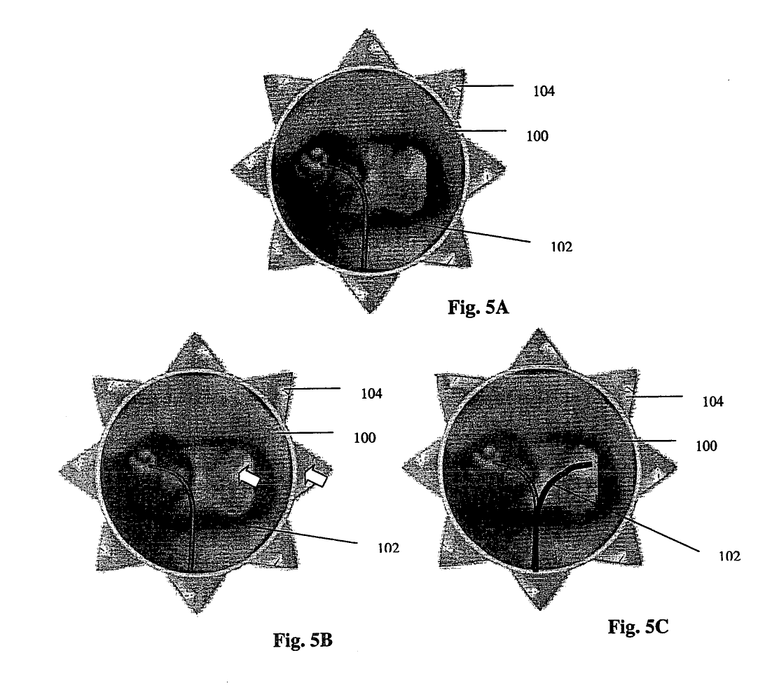

FIG. 6 is a view of a computer interface illustrating the location display method in accordance with the principles of the present invention.

Corresponding reference numerals indicate corresponding parts throughout the several views of the drawings.

### DETAILED DESCRIPTION OF THE PREFERRED EMBODIMENT

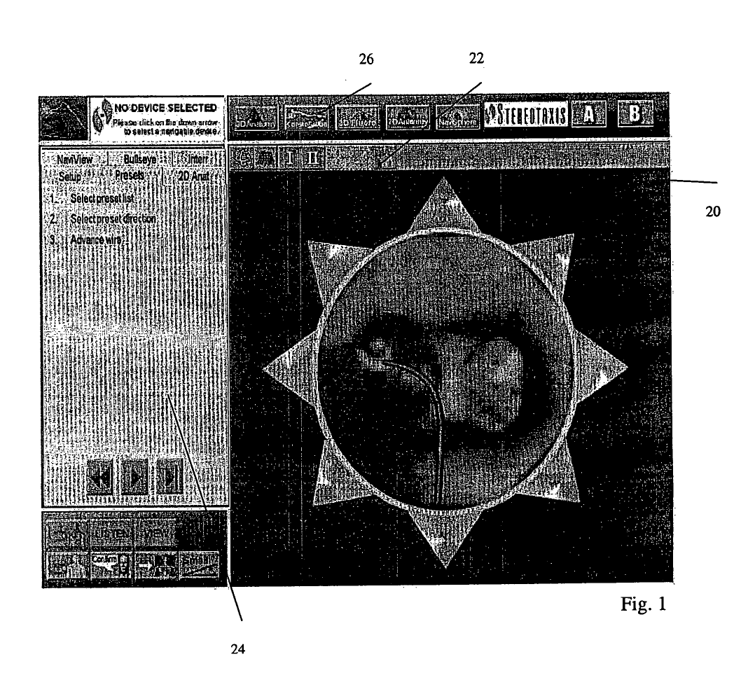

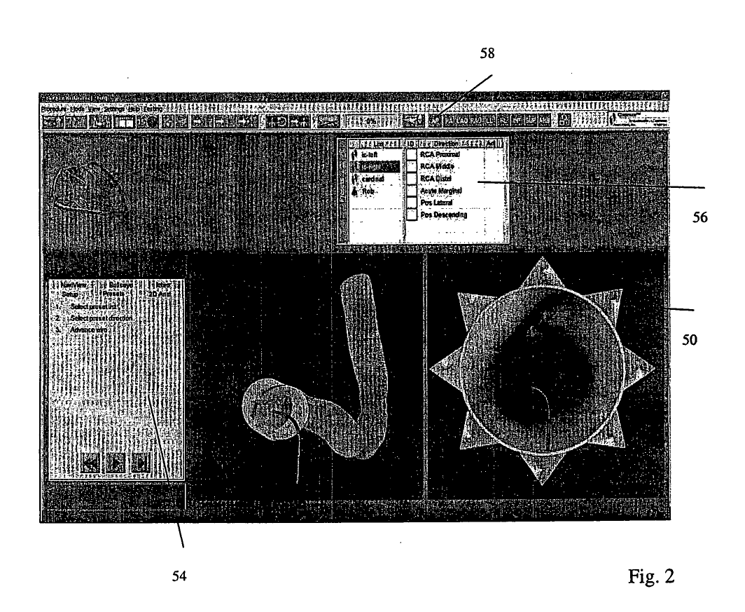

A computer screen illustrating a possible implementation of the interface and methods of the present invention is shown in FIG. 1. As shown in FIG. 1 the interface comprises a main pane 20 for implementing the interface and methods of the various embodiments of the present invention. The pane 20 can have a tool bar 22. There is an auxiliary pane 24, and a main tool bar 26. Another computer screen illustrating a possible implementation of the interface and methods of the present invention is shown in FIG. 2. As shown in FIG. 2, the interface includes main panes 50 and 52. Pane 50 is adapted for implementing the interface and methods of the various embodiments of the present invention. Pane 52 includes a display of an external view of the body lumen or cavity, to further facilitate the physician or other user understanding the position and orientation of the medical device. There is preferably an auxiliary pane 54, a navigation direction window 56, and a tool bar 58.

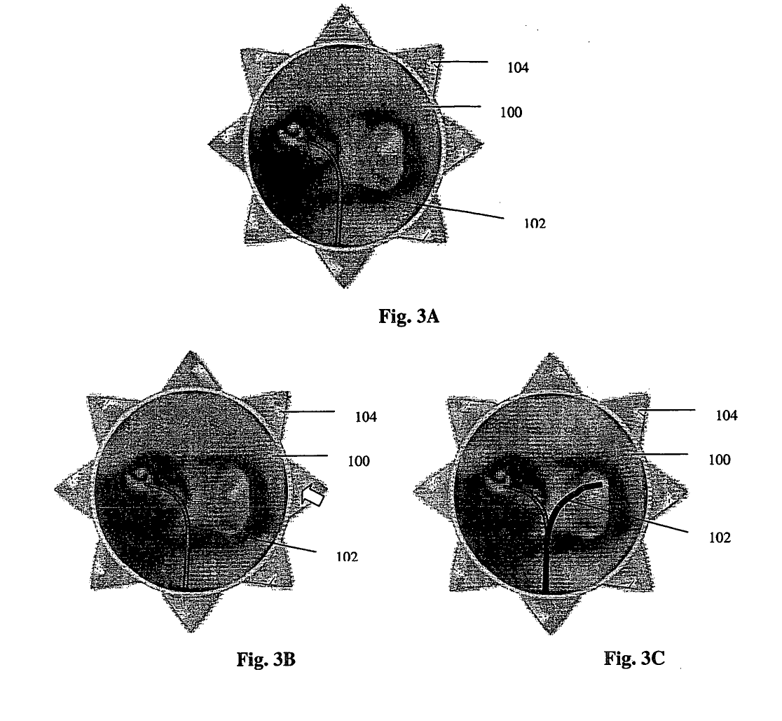

FIG. 3 shows the implementation of a first preferred embodiment of an interface and method in accordance with the principles of the present invention. The interface comprises a display 100 of an image from inside the body lumen or cavity (an “endoluminal” view) in the vicinity of the distal end of the medical device. This view may be an actual image from inside the body lumen or cavity, but in the preferred embodiments, it is a reconstructed view from preoperative or intraoperative imaging. This preoperative imaging may be x-ray imaging, M imaging, ultrasound imaging, CT imaging, rotational angiographic imaging or any other imaging modality. As shown in the Figures, the images 100 are generally circular, which is usually preferable because of the generally circular cross-section of most body lumens and cavities. However, the image 100 could have some other shape, such as oval, rectangular, or polygonal. The image preferably shows deformities, deposits, blockages and partial blockages of the vessels.

The display 100 also includes an image 102 of the distal end portion of the medical device. The image of the distal end portion of the medical device may be an actual image of the distal end portion obtained with or separately from the image of the body lumen or cavity. The image of the distal end portion of the medical device is preferably a generated image of the device based upon a model of the device and the current state of the remote navigation system. The modeling and display is disclosed in U.S. patent application Ser. No. 10/448,273, filed May 29, 2003, incorporated herein by reference. Alternatively, the orientation and or position can be determined by various localization methods, including rf localization, electrostatic localization, optical localization, ultrasound localization, or the like. In the case of navigation through a constrained lumen, such as a blood vessel, the position and orientation may be know simply from the extended length of the medical device, which is many cases is a good indicator of the position and thus the orientation of the distal end of the medical device.

The combined view of the body lumen and cavity allows the physician or other user more easily understand the current position and orientation of the medical device, and to determine the desired new direction of orientation.

In this first preferred embodiment, a plurality of control buttons 104 are associated with the displayed image. These control buttons 104 can be physical buttons. Alternatively, these control buttons 104 can be “virtual” buttons on which the physician or other user can point with a cursor or other indicator and “click”. Alternatively, these control buttons 104 can be defined locations on a touch screen display which the user can operate by touching either with a finger or a stylus. In the preferred embodiments these buttons are arranged around the periphery of the image, and the positions of the button indicate the direction they control. They may also be shaped to visually reinforce the direction associated with the particular button. As shown in FIG. 3, the buttons have a triangular shape, with the base along the perimeter of the image, and one apex of the triangle, pointing in the direction of movement that the button controls.

If the user desires to re-orient the tip of the device in a particular direction, the user simply operates the button 104 in the direction corresponding to the desired direction of movement. The displayed image 102 of the medical device updates as the remote navigation system changes the orientation of the distal end portion of the medical device. The buttons 104 can operate in a discrete mode where each operation or “click” of the button changes the orientation in the selected direction by a predetermined amount. Alternatively, or in addition, the buttons 104 could operate in a continuous mode where the direction changes as long as the button is held down.

Thus, as shown in FIG. 3B, the user manipulates a cursor to the button 104 corresponding to the desired direction of movement, and clicks. The interface instructs the remote navigation system, e.g. a Stereotaxis magnetic navigation system, or a mechanical navigation system, to change the orientation of the distal tip in the indicated direction. As shown in FIG. 3C the distal end of the device is reoriented by the remote navigation system. The user can then advance the medical device in the selected direction, or make further adjustments to the orientation of the distal end of the device.

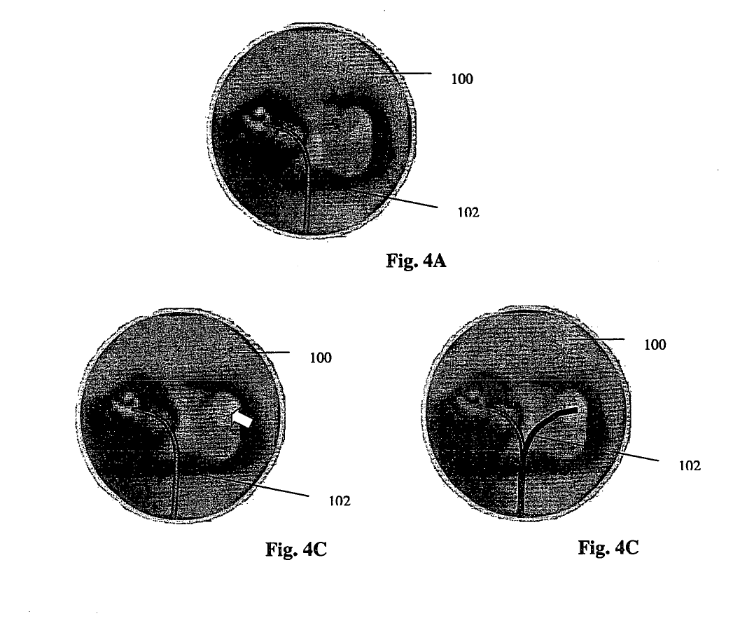

In the second preferred embodiment shown in FIG. 4, the surface of the displayed endoluminal image is active, and when the physician or user identifies a particular point on the image, the remote navigation system orients the distal tip of the device to point toward the selected point. The active surface can be one in which the user points and clicks a cursor, or alternatively it can be touch screen on which the user indicates the desired direction with a finger or a stylus. Thus as shown in FIG. 4B, the user can indicate a desired destination point on the image 100 by positioning a cursor on the desired destination and clicking. The interface instructs the remote navigation system, e.g. a Stereotaxis magnetic navigation system, or a mechanical navigation system, to change the orientation of the distal tip in the indicated direction. As shown in FIG. 4C, the distal end of the device is reoriented by the remote navigation system. The user can then advance the medical device to the selected destination, or make further adjustments to the orientation of the distal end of the device.

In the third preferred embodiment, shown in FIG. 4, the surface of the displayed endoluminal image is active, and there are also a plurality of buttons 104. The third preferred embodiment gives the user at least two alternative ways of orienting the distal end of a medical device. The user can directly control the direction of the distal tip by manipulating the buttons 104 to achieve the desired orientation. Alternatively, the user can automatically control the distal tip by picking a destination point. In either case the interface communicates the user's selection to the remote navigation system which moves the distal end of the medical device as specified. The user can then advance the medical device, or alternatively device advancement could be automatically applied by the navigation system.

To facilitate the navigation of a medical device through body lumens and cavities, it is desirable to clearly indicate to the physician or other user where the distal end of the device is presently located. Thus in accordance with one embodiment of the present invention, an external image 200 of a body lumen or cavity is displayed. The position of the medical device is determined by any conventional means of localization, including using rf signals, electrostatic localization, optical localization, image processing localization, etc. In the case of navigating through a relatively constricted lumen, such as a blood vessel, the position in the vessel can be determined by measuring the extended length of the device, as advancement of a given length will substantially correspond to the same advancement along the centerline of the vessel. The advancement of the medical device can be measured in a number of ways. If the device is advanced by machine, for example opposed rollers as disclosed in U.S. patent application Ser. No. 10/138,710, filed May 3, 2002, and U.S. patent application Ser. No. 10/858,485, filed Jun. 1, 2004, (the disclosures of which are incorporated by reference), then the rotation of the rollers can be used to measure the advancement of device. Alternatively, marks can be provided on the device which can be physically, electrically, optically, or otherwise sensed to measure the advancement of the medical device.

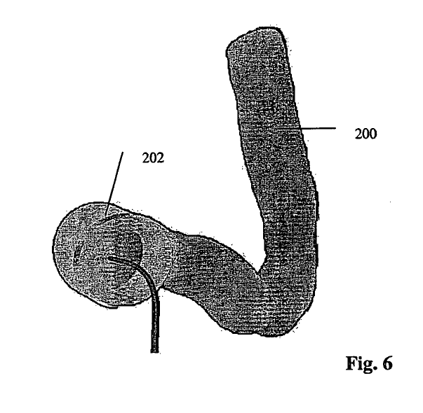

As shown in FIG. 6, a ring 202 is superimposed on the displayed image of the body lumen corresponding to the position of the distal end of the medical device. This ring is positioned in the plane perpendicular to the centerline of the lumen at the location of distal end of the medical device. The ring 202 on the image 202 helps the physician visualize the current location of the medical device.

Operation

In operation as a medical device is being navigated through a body lumen or cavity such as a blood vessel, an endoluminal view 100 of the blood vessel is displayed. The user can view the relative position of the image 102 of the medical device in the endoluminal view 100. If there is a blockage or partial blockage, the user can use the buttons 104 to adjust or suitably bias the orientation of the distal end of the device to navigate past the blockage. Alternatively, the user can click on the image 100 to adjust the orientation of the distal end of the device to navigate past the blockage. When the user reaches a branch in the blood vessel the user can navigate to and through a branch either by adjusting the orientation of the distal end of the device using buttons 104 or by adjusting the orientation of the device by pointing to the branch on the image 100 and clicking. The user interface causes the remote navigation system to change the oritentation of the distal end of the device so that it can be advanced through the vessel or other lumen or cavity, following the centerline of the path and easily steering around vascular obstructions past branches and bifurcations. To facilitate the user's operation of the interface, the interface preferably only displays and manipulate device direction.

What is claimed is:

1. A method of controlling a remote navigation system that remotely orients the distal end of the medical device in order to navigate a medical device through a body lumen, the method comprising:
displaying an endoluminal image of the portion of the body lumen through which the device is being navigated, including an image of the distal end of the medical device;
displaying a plurality of directional controls associated with the displayed endoluminal image; and
accepting inputs of a selected direction of change of orientation of the distal tip from the directional controls and in response operating the remote navigation system to change the direction of orientation of the distal end of the medical device in the selected direction.

2. The method of controlling a remote navigation system according to claim 1 wherein the endoluminal image is an actual image obtained from endoluminal imaging.

3. The method of controlling a remote navigation system according to claim 2 wherein the image of the distal end of the medical device is an actual image obtained from endoluminal imaging.

4. The method of controlling a remote navigation system according to claim 1 wherein the image of the distal end of the medial device is a generated image of the medical device.

5. The method of controlling a remote navigation system according to claim 4 wherein the image of the distal end of the medical device is generated based upon a mathematical model of the medical device.

6. The method of controlling a remote navigation system according to claim 5 wherein the image of the distal end of the medical device is generated based upon a mathematical model of the medical device and at least one control variable of the remote navigation system.

7. The method of controlling a remote navigation system according to claim 4 wherein the endoluminal image is generated from three dimensional imaging data.

8. The method of controlling a remote navigation system according to claim 7 wherein the three-dimensional imaging data is pre-operative imaging data.

9. The method of controlling a remote navigation system according to claim 7 wherein the three-dimensional imaging data is intraoperatively obtained.

10. The method of controlling a remote navigation system according to claim 6 wherein the image of the distal end of the medical device is an actual image obtained from endoluminal imaging.

11. The method of controlling a remote navigation system according to claim 6 wherein the image of the distal end of the medial device is a generated image of the medical device.

12. The method of controlling a remote navigation system according to claim 8 wherein the image of the distal end of the medical device is generated based upon a mathematical model of the medical device.

13. The method according to claim 1 wherein the step of operating the remote navigation system to change the direction of orientation of the distal end of the medical device in the selected direction comprises moving the tip in the selected direction relative to the displayed image of the lumen.

14. The method according to claim 1 wherein the step of operating the remote navigation system to change the direction of orientation of the distal end of the medical device in the selected direction comprises moving the tip in the selected direction relative to the current direction of the tip.

15. The method according to claim 1 wherein the image of the lumen is based upon a generated three-dimensional vessel path based on a reconstructed three-dimensional vessel path derived from at least a two two-dimensional x-ray images.

16. The method according to claim 1 wherein the imaging data is obtained from preoperative imaging.

17. The method according to claim 1 wherein the imaging data is obtained from intraoperative imaging.

18. The method according to claim 1 wherein the remote navigation system is a magnetic navigation system that applies a magnetic field in a selected direction in sufficient strength to orient a medical device having a magnetically responsive element.

19. The method according to claim 1 wherein the remote navigation system is a mechanical navigation system that operates a mechanism to orient the distal end of the medical device.

20. The method according to claim 1 wherein the directional control is a button.

21. The method according to claim 1 wherein the direction control is a virtual button, on which the user points and clicks.

22. The method according to claim 1 wherein the direction control is a virtual button on a pressure sensitive screen which the user pushes.

23. The method according to claim 1 wherein the distal end of the device is oriented to point toward a point that the user identified on the displayed endoluminal image.

24. A method of controlling a remote navigation system that remotely orients the distal end of the medical device in order to navigate a medical device through a body lumen, the method comprising:
displaying an endoluminal image of the portion of the body lumen through which the device is being navigated, including an image of the distal end of the medical device;
accepting inputs of a selected point on the endoluminal image and in response operating the remote navigation system to change the direction of orientation of the distal end of the medical device to point toward the selected point.

25. The method of controlling a remote navigation system according to claim 24 wherein the endoluminal image is an actual image obtained from endoluminal imaging.

26. The method of controlling a remote navigation system according to claim 24 wherein the endoluminal image is generated from three dimensional imaging data.

27. The method according to claim 26 wherein the imaging data is obtained from preoperative imaging.

28. The method according to claim 26 wherein the imaging data is obtained from intraoperative imaging.

29. The method according to claim 23 further comprising:
displaying a plurality of directional controls associated with the displayed endoluminal image; and
accepting inputs of a selected direction of change of orientation of the distal tip from the directional controls and in response operating the remote navigation system to change the direction of orientation of the distal end of the medical device in the selected direction.

30. The method of controlling a remote navigation system according to claim 29 wherein the image of the distal end of the medical device is an actual image obtained from endoluminal imaging.

31. The method of controlling a remote navigation system according to claim 29 wherein the image of the distal end of the medial device is a generated image of the medical device.

32. The method of controlling a remote navigation system according to claim 31 wherein the image of the distal end of the medical device is generated based upon a mathematical model of the medical device.

33. The method of controlling a remote navigation system according to claim 32 wherein the image of the distal end of the medical device is generated based upon a mathematical model of the medical device and at least one control variable of the remote navigation system.

34. The method of controlling a remote navigation system according to claim 29 wherein the endoluminal image is generated from three dimensional imaging data.

35. The method of controlling a remote navigation system according to claim 34 wherein the three-dimensional imaging data is pre-operative imaging data.

36. The method of controlling a remote navigation system according to claim 34 wherein the three-dimensional imaging data is intraoperatively obtained.

37. The method of controlling a remote navigation system according to claim 34 wherein the image of the distal end of the medical device is an actual image obtained from endoluminal imaging.

38. The method of controlling a remote navigation system according to claim 34 wherein the image of the distal end of the medial device is a generated image of the medical device.

39. The method of controlling a remote navigation system according to claim 38 wherein the image of the distal end of the medical device is generated based upon a mathematical model of the medical device.

40. The method according to claim 29 wherein the step of operating the remote navigation system to change the direction of orientation of the distal end of the medical device in the selected direction comprises moving the tip in the selected direction relative to the displayed image of the lumen.

41. The method according to claim 29 wherein the step of operating the remote navigation system to change the direction of orientation of the distal end of the medical device in the selected direction comprises moving the tip in the selected direction relative to the current direction of the tip.

42. The method according to claim 23 wherein the remote navigation system is a magnetic navigation system that applies a magnetic field in a selected direction in sufficient strength to orient a medical device having a magnetically responsive element.

43. The method according to claim 29 wherein the directional control is a button.

44. The method according to claim 29 wherein the direction control is a virtual button, on which the user points and clicks.

45. The method according to claim 29 wherein the direction control is a virtual button on a touch sensitive screen which the user touches.

46. A method of displaying the location of a medical device being navigated in a lumen, the method comprising:
displaying an image of the body lumen;
superimposing a ring on the displayed image of the body lumen, the ring corresponding to the plane perpendicular to the centerline of the lumen at the location of distal end of the medical device.
